(19) United States
(12) Patent Application Publication
(10) Pub. No.: US 2010/0010601 A1
(43) Pub. Date: Jan. 14, 2010

(54) Self-Aligning Latch-up Mechanism in Out of Plane Silicon Microelectrode Arrays

(76) Inventors: Sandeep Negi, Salt Lake City, UT (US); Rajmohan Bhandari, Salt Lake City, UT (US); Florian Solzbacher, Salt Lake City, UT (US); Richard A. Normann, Park City, UT (US)

Correspondence Address: THORPE NORTH & WESTERN, LLP., P.O. Box 1219, SANDY, UT, 84091-1219, US

(21) Appl. No.: 12/350,113

(22) Filed: Jan. 7, 2009

Related U.S. Application Data
(60) Provisional application No. 61/019,511, filed on Jan. 7, 2008.

(51) Int. Cl. A61N 1/05; H01L 21/3205; H01L 21/304; H01L 21/306; B32B 43/00

(52) U.S. Cl. 607/116; 438/584; 438/690; 438/700; 156/584; 257/E21.295; 257/E21.237; 257/E21.215

(57) ABSTRACT

The present invention provides microelectrode array stabilizing devices and associated methods. A microelectrode array stabilizing device includes a first microelectrode array substrate having a plurality of first microelectrodes configured to penetrate tissue. A plurality of first interlocking structures are coupled to the first microelectrode array substrate, with each of the plurality of first interlocking structures including a first interlocking mechanism at a distal end. The device may further include a second microelectrode array substrate which optionally has a plurality of second microelectrodes configured to penetrate tissue. A plurality of second interlocking structures are coupled to the second microelectrode array substrate, each of the plurality of second interlocking structures including a second interlocking mechanism at a distal end. The second interlocking mechanism is complimentary to the first interlocking mechanism. The first microelectrode array and the second microelectrode array are configured to self-align and couple together with the first interlocking mechanism secured to the second interlocking mechanism.

### RELATED APPLICATION

This application claims the benefit of U.S. Provisional Application Ser. No. 61/019,511, filed Jan. 7, 2008 and which is incorporated herein by reference.

### GOVERNMENT INTEREST

This invention was made with government support under Grant #NS042362 awarded by the National Institutes of Health and Award #N66001-05C-8045 granted by the Department of Defense/Defense Advanced Research Projects Agency. The Government has certain rights to this invention.

### BACKGROUND

The potential impact and applications for implanting electronic devices into patients via a direct interface to the neural system is vast. Systems which may enable paraplegics to regain control of their bladder or limbs, provide vision for the blind, or restore vocal cord function are all under development, and promising initial results have been obtained in some experiments.

A key component of some implantable systems is a needle array to enable interfacing of the electronics with a neuron or directly into brain tissue. For example, U.S. Pat. No. 5,215,088 to Normann et al. discloses a three-dimensional electrode device which can be used as a neural or cortical implant. The device of Norman, also known as the “Utah Electrode Array” (UEA), can be used to provide a neural interface to electronic equipment for sensing and/or stimulation.

Recent advances in micro-electro-mechanical systems (MEMS) technologies offer promising novel methods of containing neural microelectrode arrays in the targeted nerves. Such containment is necessary for chronic studies and treatment. There are several issues associated with the chronic implantation of microelectrode array, including: (1) there is substantial relative motion between the nerve and its surrounding muscles, this motion can exert forces on the microelectrode array and eventually could extract it from the nerve; (2) the nerve might be damaged if the electrodes are unable to move with the nerve. To address these issues, nerve cuff electrodes have previously been used so that they can be sized to fit snugly around a nerve but yet not restrict nerve movement.

Numerous nerve cuff electrode designs have been attempted; however, their use is not widespread because these cuffs are associated with occasional infliction of neural damage. Branner et al. [Branner A, Stein R B, Fernandez E, Aoyagi Y, Normann R A, Long-term stimulation and recording with a penetrating microelectrode array in cat sciatic nerve. IEEE Trans Biomed Eng 2004 January; 51(1):146-57] developed a containment system, which is put around the nerve and the electrode array to improve the stability of the array in the nerve. Though some functional success has been achieved in the use of these electrodes in various clinical applications, their use is not wide spread because these cuffs are associated with occasional infliction of neural damage. Also, after implantation there is some connective tissue ingrowth after a few weeks and the electrode itself does not remain in the nerve and its fascicles. The different mechanisms through which neural damage might be inflicted are (1) mechanical interaction between cuff and nerve; (2) surgical trauma to either nerve itself or its blood supply; (3) pressure caused by excessive fibrous encapsulation around the cuff; (4) the transmission of forces from adjacent muscles to the cuff and hence to the nerve; and (5) undue tension in the cuffs' leads if they are not carefully routed during implantation.

### SUMMARY

The present invention provides microelectrode array “sandwich” type devices and associated methods which avoid many of the problems associated with current approaches. In one aspect, a microelectrode array stabilizing device can include a first microelectrode array substrate having a plurality of first microelectrodes configured to penetrate tissue. A plurality of first interlocking structures can be coupled to the first microelectrode array. Each of the plurality of first interlocking structures can include a first interlocking mechanism at a distal end. The device may further include a second microelectrode array substrate, optionally having a plurality of second microelectrodes configured to penetrate tissue. A plurality of second interlocking structures can be coupled to the second microelectrode array, each of the plurality of second interlocking structures including a second interlocking mechanism at a distal end. The second interlocking mechanism can be complimentary to the first interlocking mechanism. The first microelectrode array substrate and the second microelectrode array substrate are configured to align and couple together with the first interlocking mechanism secured to the second interlocking mechanism. Also, in another form of the device, the second microelectrode array substrate may comprise just the interlocking mechanism and no electrodes. In such cases, the electrodes may be optionally retained within the interlocking mechanism such that the electrodes can be placed first followed by securing the electrodes in place using the interlocking mechanism.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention. This summary is not to be construed as limiting the claims presented subsequently insofar as this section identifies alternative embodiments of the invention and should not be used to substantively limit the broad claims unless those claims explicitly dictate such an interpretation.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 is a perspective view of a microelectrode sandwich array stabilizing device in accordance with an embodiment of the present invention.

FIGS. 2A through 2E are side schematic views of several alternative interlocking mechanisms in accordance with embodiments of the present invention.

FIG. 3A through 3F are side cross-sectional views of substrates illustrating a method of making a microelectrode sandwich array stabilizing device in accordance with another embodiment of the present invention.

FIG. 4 is a scanning electron micrograph of a microelectrode array in accordance with an embodiment of the present invention.

FIG. 5 is a scanning electron micrograph of an interlocking mechanism in accordance with an embodiment of the present invention.

FIG. 6 is a scanning electron micrograph of alignment holes in accordance with an embodiment of the present invention.

FIG. 7 is a graphical illustration of a microelectrode array stabilizing device applicator in accordance with an embodiment of the present invention.

### DETAILED DESCRIPTION OF EXAMPLE EMBODIMENTS

In describing embodiments of the present invention, the following terminology will be used.

The singular forms “a,” “an,” and “the” include plural referents unless the context clearly dictates otherwise. Thus, for example, reference to “a needle” includes reference to one or more of such needles and “etching” refers to one or more of such processing steps.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a numerical range of “50-250 micrometers” should be interpreted to include not only the explicitly recited values of about 50 micrometers and 250 micrometers, but also include individual values and sub-ranges within the indicated range. Thus, included in this numerical range are individual values such as 60, 70, and 80 micrometers, and sub-ranges such as from 50-100 micrometers, from 100-200, and from 100-250 micrometers, etc. This same principle applies to ranges reciting only one numerical value and should apply regardless of the breadth of the range or the characteristics being described.

As used herein, the term “about” means that dimensions, sizes, formulations, parameters, shapes and other quantities and characteristics are not and need not be exact, but may be approximated and/or larger or smaller, as desired, reflecting tolerances, conversion factors, rounding off, measurement error and the like and other factors known to those of skill in the art. Further, unless otherwise stated, the term “about” shall expressly include “exactly,” consistent with the discussion above regarding ranges and numerical data.

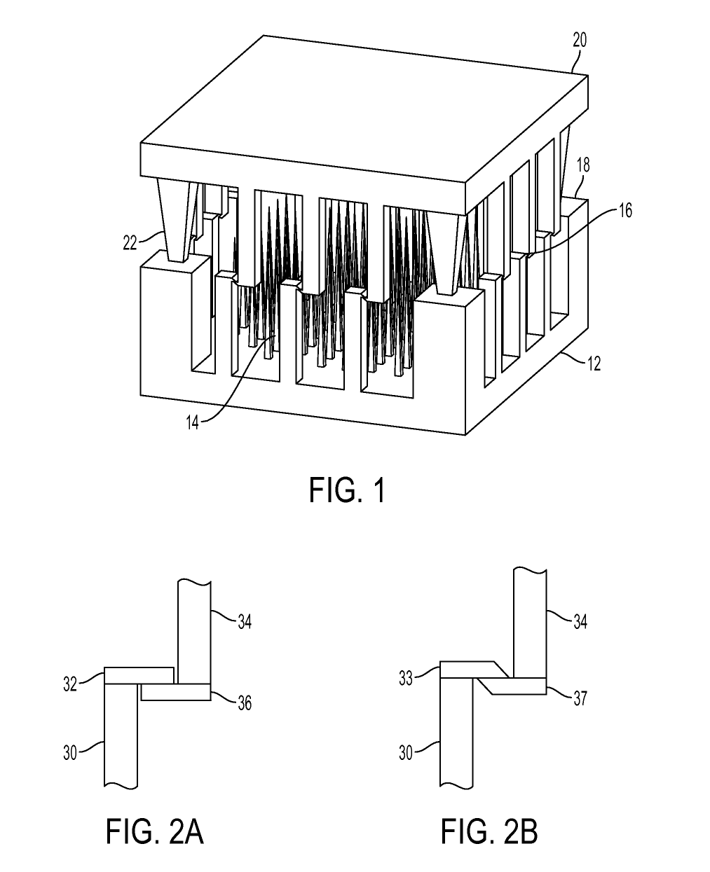

Accordingly, a novel design and fabrication method has now been discovered for a “snap-on-latch” mechanism in high density penetrating microelectrode arrays with a form fitting profile for the nerve. FIG. 1 shows a depiction of one aspect of a microelectrode sandwich array stabilizing device. A first microelectrode array substrate 12 having a plurality of first microelectrodes 14 is shown having interlocking structures with an interlocking mechanism 16. The microelectrodes may be configured in a variety of ways, depending on the desired configuration of the device and the configuration of the neural tissue. In one aspect, however, the microelectrodes may be substantially perpendicular to the microelectrode array support surface. In another aspect, the microelectrodes may be spaced apart with a substantially uniform spacing. The tips of the microelectrodes may define a planar or a non-planar tip surface. In the case of a non-planar surface, the tips of the microelectrodes vary in vertical location across the array and form a non-planar surface. This non-planar surface can be formed by microelectrodes having varying heights or uniform height microelectrodes distributed on a non-planar substrate surface. The non-planar surface may be convex, concave, slant or a more complex three-dimensional surface. Also the back/bottom side of the microelectrode array substrate (non-electrode side) can be non-planar. Such non-planar arrays can be formed as described in co-pending U.S. patent application Ser. No. 11/807,766, filed May 29, 2007.

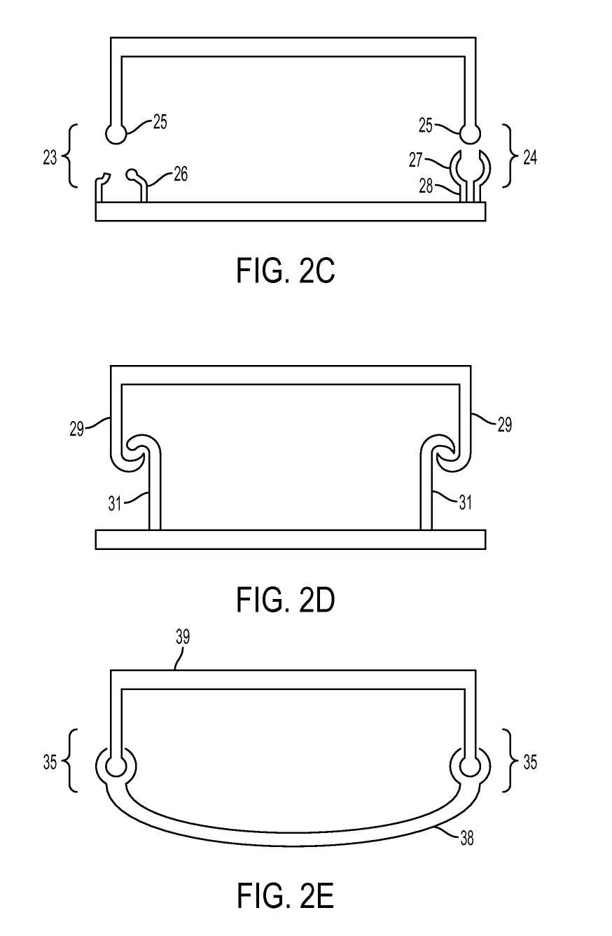

The microelectrodes can be substantially parallel to each other. This can help to avoid tissue damage when the array is inserted into tissue, since lateral displacement of tissue by the microelectrodes can be minimized by inserting the array into tissue in a direction aligned with the main axis of the microelectrodes. In contrast, a microelectrode array substrate formed by bending a flexible substrate may result in microelectrodes which are not parallel to each other. Such an array is more likely to cause tissue damage upon insertion in some applications. Furthermore, the plurality of microelectrodes formed into an array can be integrated with the substrate as a single piece during manufacture or the substrates having the interlocking mechanisms can be separately formed and then secured to the arrays. In other words, the “array substrates” of the present invention do not require the presence of an array and may merely constitute a complimentary half to a stabilizing system for a single array. The substrates can be rectangular, square or other suitable shape. For example, in FIG. 1, array substrate 20 can be rectangular, square or circular and need not be the same dimensions as the complimentary array substrate 12. Either or both of these substrates can be optionally hollow, curved (concave) to compliment peripheral nerve or other suitable shapes as further described in connection with FIGS. 2A through 2E. Further, the substrates can be hollow or solid and can have any functional curve geometry. Although the substrate can be formed of a common material from the microelectrodes, separate material can also be used such as, but not limited to, silicon, or flexible material like silicone, PDMS, parylene, and the like.

The microelectrodes may also include a conductive coating disposed on the tips of the microelectrodes to help provide a low impedance electrical connection from the tissue in which the microelectrode array is inserted to the microelectrode body. The coating may be, for example, one or more metals, designed to adhere to and provide an Ohmic contact to the material used for the microelectrode body and designed to provide a stable interface when inserted in vivo. For example, for silicon microelectrodes, a metal stack of titanium over platinum over iridium (and optionally iridium oxide) has been found to provide good performance. The titanium adheres well to silicon, the platinum provides a diffusion barrier, and the iridium provides a stable electric interface when inserted in vivo.

The microelectrode array may optionally be encapsulated in a non-conductive, non-reactive material to help improve compatibility in vivo. For example, such materials can include parylene-C, silicon carbide and/or silicone. It will be appreciated that the tips can be left un-encapsulated to allow electric contact between the microelectrodes and the tissue into which the array is inserted.

The placement of the interlocking structures can vary depending on the particular configuration of a device. In one aspect, for example, the first interlocking structures are located along at least one edge of the first microelectrode array substrate, and the second interlocking structures are located along at least one edge of the second microelectrode array substrate. The embodiment shown in FIG. 1 illustrates interlocking structures along all four edges of each substrate. However, this is not required. For example, interlocking mechanisms can be distributed along two opposing edges to allow a nerve bundle to be directed through the opposing edges which do not have interlocking mechanisms. This can reduce risk of nerve damage and yet maintain a secure stabilizing affect on the arrays relative to the nerve. In another alternative, interlocking mechanisms can be distributed within the microelectrode of the array. Additionally, the tip surface across the microelectrode arrays may also vary depending on a particular configuration or intended use. For example, in one aspect at least one of the plurality of first microelectrodes and the plurality of second microelectrodes has a non-planar tip surface. In one specific aspect, the non-planar tip surface is concave. In another specific aspect the non-planar tip surface is convex. In yet another aspect the non-planar tip surface defines a three dimensional curve.

It may be beneficial to align the first and second microelectrode array substrates relative to one another to facilitate coupling. Numerous techniques of aligning the arrays are contemplated, all of which are considered to be within the present scope of the invention. In one aspect, for example, the first microelectrode array substrate further includes at least one of a first alignment post or a first alignment hole and the second microelectrode array substrate further includes at least one of a second alignment post or a second alignment hole, respectively. The first alignment posts are configured to align with and couple to the second alignment holes, and the second alignment posts are configured to align with and couple to the first alignment holes. In another aspect, the first microelectrode array substrate includes a plurality of first alignment holes and the second microelectrode array substrate includes a plurality of second alignment posts complimentary to the first alignment holes. It is noted that although the posts and holes are illustrated as round, such features can also be varied in shape, e.g. square, rectangular, etc. Furthermore, the placement of the alignment mechanisms can vary, provided the complimentary holes and posts on each microelectrode array substrate are in alignment to allow coupling. In one example aspect, the plurality of first alignment holes are located each at a corner of the first microelectrode array, and the plurality of second alignment posts are located each at a corner of the second microelectrode array substrate in a complimentary fashion to the first alignment holes. However, any orientation and number of alignment mechanisms which directs the relative position of the first and second array substrates can be suitable. Generally, at least two such features are required to guarantee appropriate alignment.

Returning to FIG. 1, the first microelectrode array substrate 12 further includes corner alignment columns 18 having an alignment hole (not shown). A complimentary second microelectrode array substrate 20 having corner alignment posts 22 is shown facing the first microelectrode array substrate 12 and interlocking therewith at the interlocking mechanisms shown at 16. This post and hole configuration secures relative positions of each substrate via a interference fit, while the interlocking mechanisms prevent the substrates from moving apart during chronic implantation.

The interlocking mechanisms can be any complimentary features which can permanently or removably secure the first and second array substrates together. Further detail of one exemplary aspect of interlocking mechanism is shown in FIG. 2A. In this aspect, a first interlocking structure 30 having a first interlocking mechanism 32 at a distal end is shown coupled to a second interlocking structure 34 having a second interlocking mechanism 36. The interlocking structures and the interlocking mechanisms can be formed from the same material, or they may be separate materials or of separate construction. FIG. 2B shows another optional embodiment of an interlocking mechanism. In this embodiment, the first and second interlocking mechanisms 33 and 37 include an inclined surface which can allow for each member to incrementally engage the other by bending of the support shafts. In this way each interlocking mechanism can push the other temporarily aside to allow the opposite sides to snap into place. The angle of incline and the like can be adjusted depending on the flexibility of the supporting columns. Other engagement surfaces can also be used which allow each of the two complimentary mechanisms to engage the other. Other options can include providing lips or edges on the back side of the engagement surfaces to reinforce attachment once the pieces are locked together. FIG. 2C illustrates two ball-receiver interlocking mechanisms 23 and 24, with the array (or arrays) omitted. In ball-receiver 23, a ball 25 is configured in size and shape to fit inside cantilevered receiver 26. The cantilevered receiver can be a pair of members as shown having a sufficient width and/or contours to retain the ball upon engagement therewith. The receiver can generally be sufficiently flexible and elastically deform to allow the ball to enter but to also inhibit removal under normal use. Ball-receiver 24 is similar, except that receiver 27 is arcuate in shape in each of two complimentary members with a support leg 28. These types of ball-receiver mechanisms can be desirable in that they are removable so as to allow reuse of the device, repositioning of the device, or examination of the underlying nerve. FIG. 2D illustrates a hook interlocking mechanism including a pair of hooks 29 and 31 each having a hook at a distal end of a support leg which allows for flexibility as the hooks engage. As with FIGS. 2A and 2B, these hook mechanisms are generally permanent (e.g. usually require breakage of the interlocking mechanism to remove). However, the angle of incline on the hooks can be adjusted to allow for release if the support legs are sufficiently flexible. FIG. 2E illustrates a ball-receiver interlocking mechanism 35 with a contoured array substrate 38 opposite a first array substrate 39. The contoured array substrate and/or the first array substrate can be shaped to compliment a nerve bundle to reduce nerve damage or pressure. In use, one edge of ball-receivers can be engaged and then the contoured array substrate closed shut like a hinged lid to engage the opposite edge of ball-receivers. The array substrates can be formed of ceramic, plastic or other materials. Flexible plastics can be particularly suitable as they allow for flexibility and can be chosen of materials with high biocompatibility.

In another aspect of the present invention, a method of making a microelectrode array stabilizing device is provided. Such a method may include depositing a secondary layer on at least a portion of a surface of a semiconductor wafer. The secondary layer and the semiconductor wafer can be cut to form a plurality of columns. A portion of the secondary layer corresponding to intended locations of a plurality of interlocking mechanisms can be masked leaving a non-masked portion of the secondary layer. The non-masked portions of the secondary layer can then be etched away. The plurality of columns can then be etched to form microelectrodes, wherein the masked portions of the secondary layer form a plurality of interlocking mechanisms configured to couple to a complimentary facing microelectrode array.

The method may further comprise forming corner alignment columns in the semiconductor wafer. For example, an alignment hole can be ablated or etched into each of the corner alignment columns at a distal end thereof. The alignment hole can be configured to receive a complimentary alignment post from a complimentary facing microelectrode array.

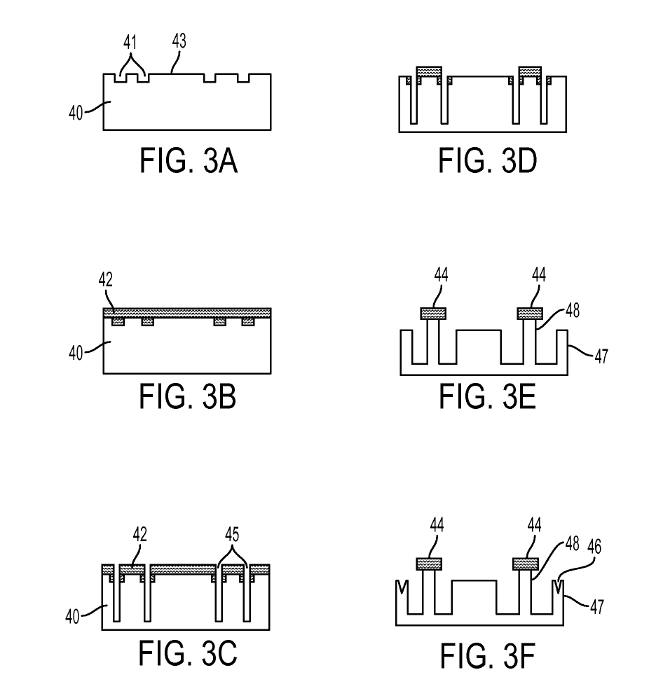

One example of a process for forming the interlocking structures and mechanisms is shown in FIG. 3A-3F. It should be noted that the materials and methods described in forming the microelectrode sandwich arrays are merely exemplary, and are not intended to be limiting. Accordingly, a semiconductor wafer 40 can be cut with shallow dicing 41 as is shown in FIG. 3A. The semiconductor wafer may be made of any material known in the semiconductor arts, such as silicon, gallium, arsenide, nitride, etc., and combinations thereof. The semiconductor wafer is then coated with a secondary layer 42 on the diced surface 43 as is shown in FIG. 3B. The secondary layer may be any material capable of forming an interlocking mechanism on a portion of the semiconductor material. One example of such a material is a nitride layer, although other materials may be suitable, e.g. chromium (around 500 nm) and oxides can also be used as a masking layer. The secondary layer can be formed by any suitable technique such as, but not limited to, chemical vapor deposition, liquid phase growth, sputtering, and the like. The secondary layer and the semiconductor layer are then deeply diced 45 to establish the structure of the intended microelectrode array, such as shown in FIG. 3C. Dicing can be accomplished by any suitable method, including laser ablation, mechanical abrading, deep reactive ion etching (DRIE), wire EDM, micro-molding, etc. In one aspect, a thin dicing blade may be used. Such methods can also be used to form the interlocking mechanisms and/or alignment mechanisms.

Following deep dicing, regions of the secondary layer that are intended to become part of the interlocking mechanism 44 are masked, and the remaining non-masked portions of the secondary layer are etched away as is shown in FIG. 3D. The remaining portions of the secondary layer form the interlocking mechanism 44 to hold the two sides of the microelectrode array sandwich together around neural tissue. The formation of the microelectrodes 48, the alignment columns 47, and the interlocking structures 44 as are shown in FIGS. 3E and 3F are described below. As shown in FIG. 3F, the alignment columns can have holes 46 or indentations into which opposing and corresponding alignment components may be inserted. Further description of methods of forming the microelectrodes can be found in U.S. patent application Ser. Nos. 11/807,766 and 11/807,764, each of which was filed on May 29, 2007, and each of which is incorporated herein by reference. Such methods can be readily applied to the present invention with the additional provision for the interlocking mechanisms and/or posts.

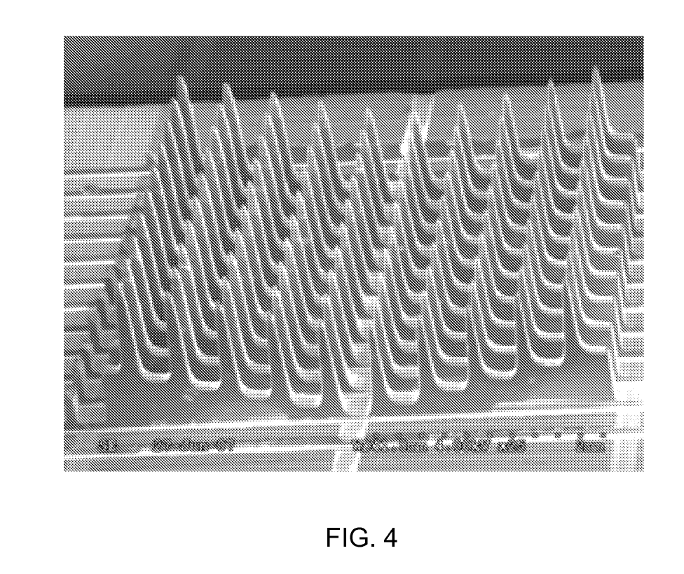

The configuration of the interlocking mechanism secures two microelectrode arrays around a nerve or portion of neural tissue together after implantation and physically stabilizes it against displacement after insertion. In one aspect, a cylindrical shape of the tips of the microelectrode arrays mate with the curved surface of the nerve. As a result the need to “seal” the cuff on the nerve trunk with suture is eliminated. FIG. 4 shows one example of a concave microelectrode array which conforms to the peripheral nerve. Such contours can be optionally formed by varying the dicing depth prior to etching.

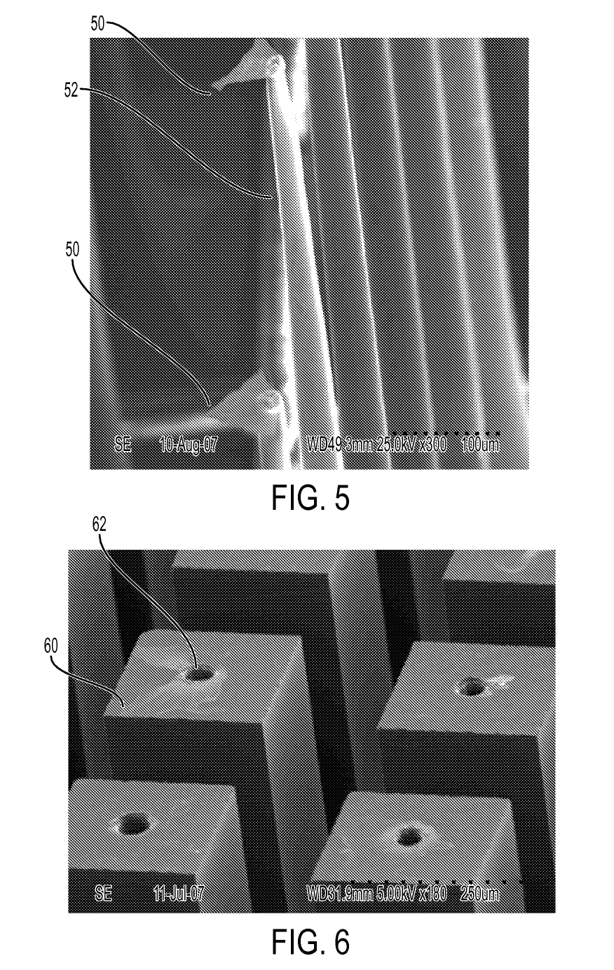

FIG. 5 shows an interlocking structure 52 having an interlocking mechanism 50 at a distal end. The interlocking mechanism was formed by masking a portion of the secondary layer as was described above. A complementary interlocking mechanism having a similar structure couples to the interlocking mechanism shown in FIG. 5 and prevents the two microelectrode arrays from coming apart due to tethering forces experienced from the nerve. In another aspect, in order to mitigate lateral movement of the assembly a “male-female” connector is also envisioned as a suitable interlocking structure. Such connectors can be similar to the post-hole alignment mechanism described below.

FIG. 6 shows one aspect of an alignment mechanism. A hole 62 is made in the distal edge columns 60 of the array. These columns can be oriented at any suitable position, although one embodiment includes such features at four edge or corner columns of one of the microelectrode arrays while a sharp electrode is made on the other microelectrode array (not shown) that is in alignment with the holes when the arrays are facing one another. As such, when the assembly is pierced through the tissue the sharp electrode sits inside the hole, thus self-aligning the two arrays and restricting relative lateral movement. To fabricate holes on the corner columns of the array, a YAG laser may be used to ablate silicon on the corner post (as in the case shown in FIG. 6). Alternately DRIE can also be used for making holes in the columns. The holes and the posts may be of any size provided they are complementary. The depth of the holes acts as a stopper so that electrodes from both the microelectrode arrays do not touch each other while snapping. In one aspect, for example, 100 μm diameter holes may be used.

Optional methods of fabricating an array of microelectrodes will now be described. Methods can include providing a semiconductor wafer from which the microelectrode array is to be constructed. The wafer can be, for example, a mono-crystalline silicon wafer. Mono-crystalline silicon wafers are typically cut from a single crystal silicon ingot, for example as for integrated circuit manufacturing. It will be appreciated that the silicon wafer need not be perfectly single crystal, as most semiconductor grade silicon wafers contain a small number of defects of various types. Other wafer materials that can be used include ceramic material and polymers.

The method further includes shaping a top surface of the wafer into a non-planar surface. It should be noted that the present scope also includes planar surfaces, and that the following discussion describes only non-planar surfaces merely for the sake of simplicity. The top surface may be shaped by gray scale lithography. A gray scale mask creates three-dimensional structures within a photo-resistant layer than can be wet or dry etched to produce a curved surface. As another example, the non-planar surface may be formed by cutting a plurality of trenches of varying depth into the top surface, wherein the depth of the trenches corresponds to a non-planar surface and etching the top surface to remove material left between the trenches to form the non-planar surface as described in further detail below. A non-planar surface may be, for example, convex, concave, slant, or other shapes.

The non-planar surface is cut into to form a plurality of trenches. Two sets of intersecting trenches are cut to form a plurality of columns having tops defined by the non-planar surface. For example, forming evenly spaced parallel saw cuts in one direction, turning the wafer 90 degrees, and forming a second set of evenly spaced parallel saw cuts, can produce a plurality of square columns. Alternately, spacing between the saw cuts can be varied to produce rectangular columns and different size square columns. More than one set of saw cuts can be used, for example, using three sets of saw cuts at 60 degrees relative to each other to form triangular or hexagonal shaped columns. Also the back/bottom side of the microelectrode array substrate (non-electrode side) can be non-planar. The cutting can be performed, for example, using a saw (e.g. a programmable dicing saw). Other techniques for cutting the trenches may also be used, including for example deep reactive ion etching.

The method also includes etching the wafer to reshape the plurality of columns to round the columns and sharpen the tops into microelectrode tips. For example, etching can be performed using a dynamic etch to round the columns and a static etch to form points at the tips of the columns.

Dynamic etching can be performed by placing the wafer into a holder and immersing the wafer in an etching solution. The holder can be constructed of a material which is resistant to the etching solution, such as polytetrafluoroethylene (e.g. Teflon®) or other polymers. The holder can include a gasket to help prevent contact between the back side of the wafer and the etching solution.

The immersed wafer can be rotated in one direction, while a stirrer stirs the etching solution in an opposite direction to provide aggressive and continuous flow of fresh etching solution into the dicing kerfs. The size of the etching bath (size of the wafer), concentration of the etching solution, total volume of the etching solution, size of the stirring bar, distance between the stirring bar and wafer, rotation rates of the wafer and stirrer, and time of etching are all interrelated. Generally, for a larger stir-bar, less rotation of the stir bar is required to create a desirable vortex which drives a strong flush of etchant into the columns. For example, using an etching container of 7.5 inch diameter, a stirring bar of 3 inches length, and 2 inches of separation between the stirrer bar and wafer, it was found that rotating the wafer holder at about 22 rpm while counter rotating the stirrer bar at about 500 rpm provided good uniformity of the etching of the columns. Using a solution of 5% hydrofluoric acid (49% concentration) and 95% nitric acid (69% concentration), etching time of about 4 minutes reduces 250 micrometer square columns to round column of about 150 micrometers in diameter. After the dynamic etching, the columns have been reshaped to a plurality of rounded columns having substantially uniform cross section from base to tip, although there may be some narrowing of the columns toward the tip.

Static etching to sharpen the tips can be performed by inverting the holder to position the wafer columns in an upward direction and placing the wafer into etchant solution which is not stirred. In the static etching, the etching at the tips of the columns is faster than near the bases, because little fluid motion is present to replenish etching solution near to bottoms of the kerfs. This preferentially shapes the columns into microelectrodes. Using the same etching solution as described above, static etching can be performed for about 8 minutes. Longer etching tends to form more sharply pointed microelectrodes, while shorter etching time tends to form more rounded, missile-shaped microelectrodes. Etching time may be in the range of about 2 to about 20 minutes, although in general longer etching times tend to decrease uniformity.

The depth of the sawing can be constant so that the bottom of the trenches defines a plane. The bases of the microelectrodes are thus disposed within a plane, and the micro-microelectrodes have varying length (height).

Alternately, the depth of the cutting can be varied so that the bases of the columns define a second non-planar surface. The depth of the cutting can be varied during forming of one set of parallel trenches to define a one-dimensional curve, or the depth can be varied during both sets of parallel trenches to define a two-dimensional curve. If the depth of the cutting follows the same profile as the (upper) non-planar surface, the microelectrodes will have substantially the same height.

Generally the microelectrodes can be electrically insulated from each other. This can be accomplished by cutting a third set of trenches into a back surface of the wafer, and filling the third set of trenches with an electrically insulating material. This operation can be performed before cutting the first set and the second set of trenches into the top side of the wafer. The first set and the second set of trenches can be cut sufficiently deeply into the top side of the wafer to intersect the third set of trenches (reaching the insulating material), thus removing all of the original wafer material between the columns that will be formed into the microelectrodes. The insulating material can be, for example, glass. A glass frit can be disposed into the third set of trenches and then heating to melt and anneal the glass.

As another example, a polymer can be reflowed into the trenches. For example, biocompatible polymer, such as but not limited to benzo-cyclobutane (BCB), can be also be used as an insulating material between the electrodes. BCB can be selectively patterned on the trenches using standard lithographic techniques (e.g. spin coating and curing).

In yet another aspect of the present invention, a method of using a microelectrode array “sandwich” type device may include applying the one microelectrode array to a first side of a portion of neural tissue. The second microelectrode array can be applied to an opposite side of the portion of neural tissue such that the plurality of first microelectrodes is facing the second microelectrode substrate. The portion of neural tissue can be positioned there between. The first microelectrode array substrate and the second microelectrode array substrate can be pushed together sufficient to allow the alignment posts to align with the alignment holes and the first interlocking mechanism to couple to the second interlocking mechanism.

As will be appreciated, the arrays and latching mechanisms of the present invention are most often relatively small, e.g. less than about 10-15 mm across, making manual positioning a challenge. In one aspect of the present invention, a mechanism for positioning the microelectrode array “sandwich” type device is provided. Such a device may include a first support member configured to couple to the first microelectrode array substrate. A second support member can be configured to couple to the second microelectrode array substrate and to position the second microelectrode array substrate in a position complimentary to and facing the first microelectrode array substrate. A springing member can be coupled to the first support member and the second support member. The springing member can be configured to move the first microelectrode array substrate and the second microelectrode array substrate together in an aligned orientation. Such a spring member can be integral with the first and second support members or be formed of a separate material which is coupled thereto. A first uncoupling mechanism can be disposed on the first support member and a second uncoupling mechanism disposed on the second support member. The first uncoupling mechanism and the second uncoupling mechanism can be configured to release the first and second microelectrode arrays upon coupling.

A variety of uncoupling mechanisms are contemplated, and any mechanism capable of releasing the microelectrode array once coupled in the neural tissue should be considered to be within the present scope. In one aspect, for example, at least one of the first uncoupling mechanism and the second uncoupling mechanism includes an electrostatic interaction. An applied potential can be used to vary electrostatic strength. In another aspect, at least one of the first uncoupling mechanism and the second uncoupling mechanism includes a capillary interaction, e.g. an evaporable liquid place between the support members and the stabilizing device. Optionally, an applied potential can be used to vary electrowetting strength of the surfaces. In one aspect, one of the first uncoupling mechanism and the second uncoupling mechanism may include vacuum. In yet another aspect, at least one of the first uncoupling mechanism and the second uncoupling mechanism includes an adhesive interaction.

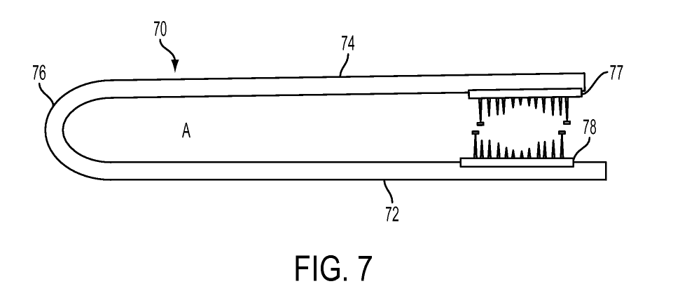

In another aspect of the present invention, a stapler-like device may be utilized to insert the sandwich array into neural tissue. FIG. 7 shows one example of such a device. The device 70 may be configured to have a first supporting member 74 configured to couple to the first microelectrode array 77, and a second support member 72 configured to couple to the second microelectrode array 78 and to position the second microelectrode array in a position complimentary to and facing the first microelectrode array. A springing member 76 is coupled to the first support member and the second support member, and is configured to move the first microelectrode array and the second microelectrode array together in an aligned orientation. The device may further include a first uncoupling mechanism disposed on the first support member and a second uncoupling mechanism disposed on the second support member, where the first uncoupling mechanism and the second uncoupling mechanism are configured to release the first and second microelectrode arrays upon coupling.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention.

1. A microelectrode array stabilizing device, comprising:
a first microelectrode array substrate having a plurality of first microelectrodes associated therewith and configured to penetrate tissue;
a plurality of first interlocking structures coupled to the first microelectrode array, each of the plurality of first interlocking structures including a first interlocking mechanism at a distal end;
a second microelectrode array substrate; and
a plurality of second interlocking structures coupled to the second microelectrode array, each of the plurality of second interlocking structures including a second interlocking mechanism at a distal end, the second interlocking mechanism being complimentary to the first interlocking mechanism;
wherein the first microelectrode array substrate and the second microelectrode array substrate are configured to align and couple together with the first interlocking mechanism secured to the second interlocking mechanism.

2. The device of claim 1, wherein the first interlocking structures are located along at least one edge of the first microelectrode array substrate, and the second interlocking structures are located along at least one edge of the second microelectrode array substrate.

3. The device of claim 1, wherein at least one of the plurality of first microelectrodes and the plurality of second microelectrodes has a non-planar tip surface.

4. The device of claim 3, wherein the non-planar tip surface is concave.

5. The device of claim 3, wherein the non-planar tip surface defines a three dimensional curve.

6. The device of claim 1, wherein the second microelectrode array substrate further includes a plurality of second microelectrodes configured to penetrate tissue.

7. The device of claim 1, wherein one or both of the first microelectrode array substrate and the second microelectrode array substrate have a non-planar back side.

8. The device of claim 1, wherein the first microelectrode array substrate further includes at least one of a first alignment post or a first alignment hole and the second microelectrode array substrate further includes at least one of a second alignment post or a second alignment hole, wherein the first alignment posts are configured to self-align with and couple to the second alignment holes, and the second alignment posts are configured to align with and couple to the first alignment holes.

9. The device of claim 8, wherein the first microelectrode array substrate includes a plurality of first alignment holes and the second microelectrode array substrate includes a plurality of second alignment posts complimentary to the first alignment holes.

10. The device of claim 9, wherein the plurality of first alignment holes are located each at a corner of the first microelectrode array, and the plurality of second alignment posts are located each at a corner of the second microelectrode array.

11. The device of claim 1, wherein the first interlocking mechanism and the second interlocking mechanism are configured to couple together in a hooking fashion.

12. The device of claim 1, wherein the plurality of first interlocking structures are coupled to the first microelectrode array substrate and the plurality of second interlocking structures are coupled to the second microelectrode array substrate as separate non-integral pieces.

13. A method of making a microelectrode array stabilizing device, comprising:
depositing a secondary layer on at least a portion of a surface of a semiconductor wafer;
cutting the secondary layer and the semiconductor wafer to form a plurality of columns;
masking a portion of the secondary layer corresponding to intended locations of a plurality of interlocking mechanisms and leaving a non-masked portion of the secondary layer;
etching away the non-masked portions of the secondary layer; and
etching the plurality of columns to form microelectrodes, wherein the masked portions of the secondary layer form a plurality of interlocking mechanisms configured to couple to a complimentary facing microelectrode array.

14. The method of claim 13, further comprising:
forming corner alignment columns in the semiconductor wafer; and
ablating or etching to create an alignment hole into each of the corner alignment columns at a distal end thereof, the alignment hole being configured to receive a complimentary alignment post from a complimentary facing microelectrode array.

15. The method of claim 13, wherein the secondary layer is a nitride, chromium or oxide layer.

16. The method of claim 13, wherein the semiconductor wafer is silicon.

17. A method of using the microelectrode array stabilizing device of claim 1, comprising;
applying the plurality of first microelectrodes to a first side of a portion of neural tissue;
applying the second microelectrode array substrate to an opposite side of the portion of neural tissue such that the plurality of first microelectrodes is facing the second microelectrode array substrate and the portion of neural tissue is positioned therebetween; and
pushing the first microelectrode array substrate and the second microelectrode array substrate together sufficient to allow the first interlocking mechanism to couple to the second interlocking mechanism.

18. A mechanism for positioning the microelectrode array stabilizing device of claim 1, comprising:
a first support member configured to couple to the first microelectrode array substrate;
a second support member configured to couple to the second microelectrode array substrate and to position the second microelectrode array substrate in a position complimentary to and facing the first microelectrode array substrate;
a springing member coupled to the first support member and the second support member, the springing member configured to move the first microelectrode array substrate and the second microelectrode array substrate together in an aligned orientation; and
a first uncoupling mechanism disposed on the first support member and a second uncoupling mechanism disposed on the second support member, the first uncoupling mechanism and the second uncoupling mechanism configured to release the first and second microelectrode array substrates upon coupling.

19. The device of claim 18, wherein at least one of the first uncoupling mechanism and the second uncoupling mechanism includes an electrostatic interaction.

20. The device of claim 18, wherein at least one of the first uncoupling mechanism and the second uncoupling mechanism includes a capillary interaction.

21. The device of claim 18, wherein at least one of the first uncoupling mechanism and the second uncoupling mechanism includes an adhesive interaction or a vacuum.

22. The device of claim 18, wherein the first support member, the second support member, and the springing member are continuously formed of a single structure.
